(19) United States
(12) Patent Application Publication
(10) Pub. No.: US 2019/0144545 A1
(43) Pub. Date: May 16, 2019

(54) PD-1/PD-L1 Inhibitors for Cancer Treatment

(71) Applicants: MERCK PATENT GMBH, Darmstadt (DE); PFIZER INC., New York, NY (US)

(72) Inventors: Dimitry NUYTEN, San Francisco, CA (US); Alexei MOROZOV, New York, NY (US); Adrian WOOLFSON, Brooklyn, NY (US); Aron THALL, San Diego, CA (US); Kevin CHIN, Sudbury, MA (US); Satjit BRAR, San Diego, CA (US)

(73) Assignees: MERCK PATENT GMBH, Darmstadt (DE); PFIZER INC., New York, NY (US)

(21) Appl. No.: 16/304,338

(22) PCT Filed: May 22, 2017
(86) PCT No.: PCT/EP2017/062213

§ 371 Date: Nov. 26, 2018

Related U.S. Application Data
(60) Provisional application No. 62/341,921, filed on May 26, 2016.
(60) Provisional application No. 62/423,358, filed on Nov. 17, 2016.
(60) Provisional application No. 62/471,459, filed on Mar. 15, 2017.

(51) Int. Cl. C07K 16/28; A61P 35/04

(52) U.S. Cl. CPC C07K 16/2827; A61P 35/04; A61K 45/06; C07K 2317/565; C07K 2317/76; C07K 2317/21

(57) ABSTRACT

The invention relates to methods of treating cancer in a subject, comprising administering to the subject a therapeutically effective amount of an inhibitor of the interaction between the PD-1 receptor and its ligand PD-L1.

The invention relates to methods of treating cancer in a subject, comprising administering to the subject a therapeutically effective amount of an inhibitor of the interaction between the PD-1 receptor and its ligand PD-L1.

### BACKGROUND OF THE INVENTION

### Cancer

Cancer is an abnormal growth of cells which tend to proliferate in an uncontrolled way and, in some cases, to metastasize (spread). Cancer is not one disease. It is a group of more than 100 different and distinctive diseases. Cancer can involve any tissue of the body and have many different forms in each body area. Most cancers are named for the type of cell or organ in which they start. If a cancer spreads (metastasizes), the new tumor bears the same name as the original (primary) tumor. The frequency of a particular cancer may depend on gender. While skin cancer is the most common type of malignancy for both men and women, the second most common type in men is prostate cancer and in women, breast cancer.

### Ovarian Cancer

For women globally, ovarian cancer is the seventh most common cancer and the eighth leading cause of cancer death (Globocan Population Fact Sheet 2012). In the United States, the age-standardized incidence rate (ASR) based on 2007-2011 cases was 12.3 per 100,000 women, which represents an increase from an estimated ASR of 8.1 per 100,000 based on 2000-2009 cases. Because the disease lacks perceptible symptoms at an early stage, patients typically present with advanced disease.

The 5-year survival rate ranges from approximately 30% to 50% (SEER Stat Fact Sheet Ovary Cancer 2014). The addition of paclitaxel to platinum-based chemotherapy improved both progression-free survival (PFS) and overall survival (OS) in patients with advanced disease. Antiangiogenic agents, such as bevacizumab and pazopanib, have been shown to prolong PFS, but not OS.

PARP inhibitors (eg, olaparib) added to chemotherapy have shown promise, but are predominately used in the maintenance setting. The majority of patients experience relapse, typically related to platinum resistance, thus making ovarian cancer an often fatal disease with few approved or effective treatment options (Luvero D, et al. Ther Adv Med Oncol. 2014; 6(5):229-239).

### Renal Cell Carcinoma

Renal cell carcinoma (RCC) is the most common kidney cancer and constitutes about 3% of all malignant tumors in adults. Until 2005, interferon-alpha (IFN-α) and high-dose interleukin (IL)-2 therapies were the standards of care for patients with advanced RCC (aRCC), albeit with modest efficacy. Since then, development and approval of multiple vascular endothelial growth factor (VEGF) pathway and mammalian target of rapamycin (mTOR) inhibitors have significantly improved the outcomes of aRCC patients. These agents include the VEGF receptor (VEGFR) tyrosine kinase inhibitors (TKIs) sunitinib, pazopanib, axitinib and sorafenib, the mTOR inhibitors temsirolimus and everolimus, and the anti-VEGF monoclonal antibody bevacizumab. However, despite the substantial improvement of patient outcomes with these agents, durable and complete responses in aRCC patients are uncommon; the majority of patients will eventually develop resistance, exhibit disease progression while on therapy, and succumb to death due to metastatic disease.

### Hodgkin's Lymphoma

Lymphoma is the most common blood cancer. The two main forms of lymphoma are Hodgkin lymphoma (HL) and non-Hodgkin lymphoma (NHL). Lymphoma occurs when cells of the immune system called lymphocytes, a type of white blood cell, grow and multiply uncontrollably. Cancerous lymphocytes can travel to many parts of the body, including the lymph nodes, spleen, bone marrow, blood, or other organs, and form a mass called a tumor. The body has two main types of lymphocytes that can develop into lymphomas: B-lymphocytes (B-cells) and T-lymphocytes (T-cells). HL, also known as Hodgkin disease, is not as common as NHL. Approximately 9,000 new cases of HL are projected each year. Although HL can occur in both children and adults, it is most commonly diagnosed in young adults between the ages of 20 and 34 years.

HL is characterized by the presence of very large cells called Reed-Sternberg (RS) cells, although other abnormal cell types may be present. HL usually starts in the lymph nodes; however, it often spreads from one lymph node to another and can also spread to other organs.

Common signs and symptoms of HL include swelling of the lymph nodes (which is often but not always painless), fever, night sweats, unexplained weight loss, and lack of energy. While most people who have these complaints will not have HL, anyone with persistent symptoms should be seen by a physician to make sure that lymphoma is not present.

HL has been divided into two main classifications: classical HL (CHL), which accounts for 90 to 95 percent of cases, and nodular lymphocyte predominant HL. The type of HL a patient has may affect their treatment choices.

### Classical Hodgkin Lymphoma

Nodular Sclerosis CHL is the most common subtype of HL, accounting for 60 to 80 percent of all HL cases. In nodular (knot-like) sclerosis CHL, the involved lymph nodes contain RS cells mixed with normal white blood cells. The lymph nodes often contain a lot of scar tissue, which is where the name nodular sclerosis (scarring) originates. The disease is more common in women than in men, and it usually affects adolescents and adults under the age of 50. The majority of patients are cured with current treatments.

Mixed Cellularity CHL accounts for about 15 to 30 percent of all HL cases. The disease is found more commonly in men than in women, and it primarily affects older adults. With this type of CHL, the lymph nodes contain many RS cells in addition to several other cell types. More advanced disease is usually present by the time this subtype is diagnosed.

Lymphocyte-Depletion CHL is rarely diagnosed. Abundant RS cells and few normal lymphocytes are present in the lymph nodes of patients with this subtype, which is aggressive and usually not diagnosed until it is widespread throughout the body. Lymphocyte-Rich CHL accounts for less than five percent of HL cases. The disease may be diffuse (spread out) or nodular in form and is characterized by the presence of numerous normal-appearing lymphocytes and classic RS cells. This subtype of HL is usually diagnosed at an early stage in adults and has a low relapse (disease returns after treatment) rate.

### Lymphocyte Predominant Hodgkin Lymphoma

Nodular Lymphocyte Predominant HL accounts for five to 10 percent of all HL cases. It affects men more often than women and is usually diagnosed before the age of 35. In nodular lymphocyte predominant HL, most of the lymphocytes found in the lymph nodes are normal (not cancerous). Typical RS cells are usually not found in this subtype, but large, abnormal B cells (sometimes referred to as popcorn cells) can be seen as well as small B cells, which may be distributed in a nodular pattern within the tissues. This subtype is usually diagnosed at an early stage and is not very aggressive. In many ways, this form of HL resembles indolent (slow-growing) B-cell NHL with late recurrences.

(source: http:www.lymphoma.org)

### Head and Neck Squamous Cell Carcinoma (HNSCC)

In 2016, it is estimated that 61,760 individuals will be diagnosed with head and neck cancer in the United States, with approximately 13,190 deaths from the disease. Most patients with head and neck cancer have metastatic disease at the time of diagnosis (regional nodal involvement in 43% and distant metastasis in 10%). Head and neck cancers encompass a diverse group of uncommon tumors that frequently are aggressive in their biologic behavior. Moreover, patients with a history of head and neck cancer have the potential to develop a second primary tumor, generally due to the habitual use of tobacco.

These new primary tumors occur at an annual rate of 3% to 7%, and 50% to 75% of such new cancers occur in the upper aerodigestive tract or lungs. The incidence of tobacco-related head and neck cancer is decreasing. However, the incidence of cancer due to the human papillomavirus (HPV) continues to increase at a rate of 2 to 4% per year.

(source: http://www.cancernetwork.com)

### BRIEF DESCRIPTION OF THE FIGURES

FIG. 1a (SEQ ID NO:7) shows the full length heavy chain sequence of Avelumab.

FIG. 1b (SEQ ID NO:8) shows the heavy chain sequence of Avelumab without the C-terminal lysine.

FIG. 2 (SEQ ID NO:9) shows the light chain sequence of Avelumab.

### GENERAL DESCRIPTION OF THE INVENTION

As there still is a high unmet medical need regarding the treatment of the before mentioned cancer types, it is an aspect of the present invention to provide a method of treating these cancer types in a subject, comprising administering to the subject a therapeutically effective amount of an inhibitor of the interaction between the PD-1 receptor and its ligand PD-L1.

Specific types of cancer to be treated according to the invention include, but are not limited to, ovarian cancer, renal cell carcinoma, or Hodgkin's lymphoma, which cancers may be untreated or previously treated, primary or metastatic, refractory, or recurrent.

In one embodiment of the invention the subject is human, the PD-1 receptor is human PD-1 receptor, and PD-L1 is human PD-L1.

In a preferred embodiment of the invention the inhibitor binds to PD-L1.

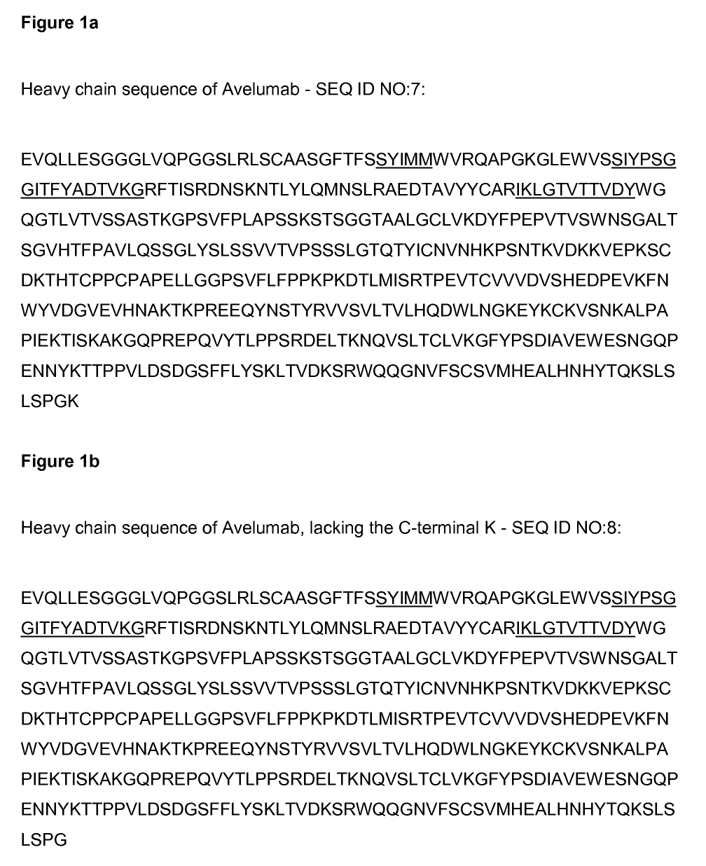

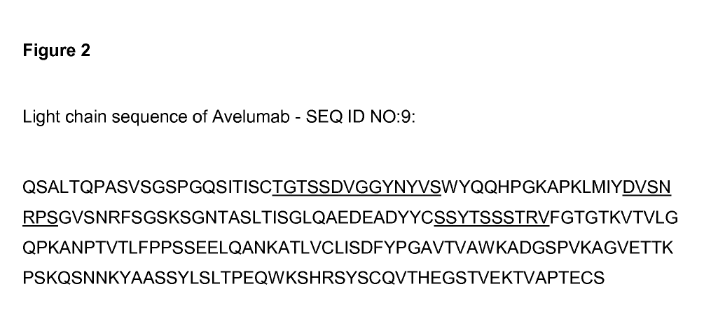

In a more preferred embodiment the inhibitor is an anti-PD-L1 antibody. In some embodiments, the anti-PD-L1 antibody comprises three complementarity determining regions (CDRs) (SEQ ID NOs: 1, 2 and 3) from the heavy chain amino acid sequence shown in FIG. 1a (SEQ ID NO:7) and 1b (SEQ ID NO:8), and three CDRs (SEQ ID NOs: 4, 5 and 6) from the light chain amino acid sequence shown in FIG. 2 (SEQ ID NO:9), as marked by underlining, and described in further detail in WO2013079174. In a more preferred embodiment, the anti-PD-L1 antibody is Avelumab, having the heavy and light chain sequences shown in FIG. 1a or 1b and 2 (SEQ ID NOs: 7 or 8, and 9).

FIG. 1a (SEQ ID NO:7) shows the full length heavy chain sequence of Avelumab. It is frequently observed, however, that in the course of antibody production the C-terminal lysine (K) of the heavy chain is cleaved off. This modification has no influence on the antibody-antigen binding. Therefore, in some embodiments the C-terminal lysine (K) of the heavy chain sequence of Avelumab is absent. The heavy chain sequence of Avelumab without the C-terminal lysine is shown in FIG. 1b (SEQ ID NO:8).

In another embodiment of the invention the anti-PD-L1 antibody is administered at a dose of 10 mg/kg body weight every other week (i.e. every two weeks, or “Q2W”)).

In one embodiment, the method results in an objective response, preferably a complete response or partial response in the subject.

In one embodiment, the inhibitor is administered intravenously (e.g. as an intravenous infusion) or subcutaneously. Preferably, the inhibitor is administered as an intravenous infusion. More preferably, the inhibitor is administered as a one hour intravenous infusion.

In one embodiment the inhibitor is administered as a single agent, i.e. not as part of a combination therapy.

In one aspect, the cancer is ovarian cancer.

In one embodiment the subject having ovarian cancer has not been previously treated for ovarian cancer, i.e. the ovarian cancer has not previously been treated.

In one embodiment the subject having previously untreated ovarian cancer is receiving the inhibitor in combination with chemotherapy.

In one embodiment the subject having previously untreated ovarian cancer is receiving the inhibitor following chemotherapy.

In a further embodiment said chemotherapy is platinum-based chemotherapy.

In a further aspect, the cancer is renal cell carcinoma.

In one embodiment the renal cell carcinoma is metastatic renal cell carcinoma.

In one embodiment the metastatic renal cell carcinoma has previously received systemic treatment.

In one embodiment the renal cell carcinoma is treated with the inhibitor as a single agent, i.e. not as part of a combination therapy.

In a further aspect, the cancer is Hodgkin's lymphoma.

In one embodiment the Hodgkin's lymphoma is classical Hodgkin's lymphoma.

In one embodiment the Hodgkin's lymphoma is advanced stage.

In one embodiment the Hodgkin's lymphoma has previously received chemotherapy.

In a further aspect, the cancer is head and neck squamous cell carcinoma (HNSCC).

In one embodiment the HNSCC is metastatic.

In one embodiment the HNSCC has previously received chemotherapy comprising a platinum containing chemotherapeutic agent.

In one embodiment the HNSCC is platinum-refractory.

In one embodiment the HNSCC is platinum-ineligible.

In one embodiment the HNSCC is metastatic, and platinum-refractory or platinum-ineligible.

Also provided is the use of an anti-PD-L1 antibody in the manufacture of a medicament for the treatment of cancer in an individual. Also provided is an anti-PD-L1 antibody for use in the treatment of cancer.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact polyclonal or monoclonal antibodies, but also, unless otherwise specified, any antigen binding fragment thereof that competes with the intact antibody for specific binding, fusion proteins comprising an antigen binding portion (e.g., antibody-drug conjugates), any other modified configuration of the immunoglobulin molecule that comprises an antigen recognition site, antibody compositions with polyepitopic specificity, multispecific antibodies (e.g., bispecific antibodies).

Antigen binding fragments include, for example, Fab, Fab′, F(ab′)₂, Fd, Fv, domain antibodies (dAbs, e.g., shark and camelid antibodies), fragments including complementarity determining regions (CDRs), single chain variable fragment antibodies (scFv), maxibodies, minibodies, intrabodies, diabodies, triabodies, tetrabodies, v-NAR and bis-scFv, and polypeptides that contain at least a portion of an immunoglobulin that is sufficient to confer specific antigen binding to the polypeptide.

The term “immunoglobulin” (Ig) is used interchangeably with “antibody” herein. The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for p and c isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8th Edition, Daniel P. Sties, Abba I. Terr and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated α, δ, ε, γ and ρ, respectively. The γ and α classes are further divided into subclasses on the basis of relatively minor differences in the CH sequence and function, e.g., humans express the following subclasses: IgG1, IgG2A, IgG2B, IgG3, IgG4, IgA1 and IgK1.

An “isolated” antibody is one that has been identified, separated and/or recovered from a component of its production environment (E.g., natural or recombinant). Preferably, the isolated polypeptide is free of association with all other components from its production environment. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (1) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant cells since at least one component of the antibody's natural environment will not be present. Ordinarily, however, an isolated polypeptide or antibody will be prepared by at least one purification step.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al, Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al, Hybridoma, 14 (3): 253-260 (1995), Harlow et al, Antibodies: A Laboratory Manual, (Cold Spring Harbor Laboratory Press, 2^{nd} ed. 1988); Hammerling et al, in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al, Nature, 352: 624-628 (1991); Marks et al, J. Mol Biol. 222: 581-597 (1992); Sidhu et al, J. Mol Biol. 338(2): 299-310 (2004); Lee et al, J. Mol Biol. 340(5): 1073-1093 (2004); Fellouse, Proc. Natl. Acad. ScL USA 101(34): 12467-12472 (2004); and Lee et al, J. Immunol. Methods 284(1-2): 119-132 (2004), and technologies for producing human or humanlike antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al, Proc. Natl. Acad. ScL USA 90: 2551 (1993); Jakobovits et al, Nature 362: 255-258 (1993); Bruggemann et al, Year in Immunol. 7:33 (1993); U.S. Pat. Nos. 5,545,807; 5,545,806; 5,569,825; 5,625,126; 5,633,425; and U.S. Pat. No. 5,661,016; Marks et al, Bio/Technology 10: 779-783 (1992); Lonberg et al, Nature 368: 856-859 (1994); Morrison, Nature 368: 812-813 (1994); Fishwild et al, Nature Biotechnol 14: 845-851 (1996); Neuberger, Nature Biotechnol. 14: 826 (1996); and Lonberg and Huszar, Intern. Rev. Immunol. 13: 65-93 (1995).

An “antigen binding fragment” of an antibody, or “antibody fragment” comprises a portion of an intact antibody, which is still capable of antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al, Protein Eng. 8HO): 1057-1062 [1995]); single-chain antibody molecules and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site. “Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994). “Functional fragments” of the antibodies of the invention comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al, Proc. Natl. Acad. ScL USA 90: 6444-6448 (1993).

The term “nanobodies” refers to single-domain antibodies which are fragments consisting of a single monomeric variable antibody domain. Like a whole antibody, they are able to bind selectively to a specific antigen. With a molecular weight of only 12-15 kDa, single-domain antibodies are much smaller than common antibodies (150-160 kDa). The first single-domain antibodies were engineered from heavy-chain antibodies found in camelids. Gibbs, W. Wayt (August 2005). “Nanobodies”. Scientific American Magazine.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al, Proc. Natl. Acad. ScL USA, 81:6851-6855 (1984)). As used herein, “humanized antibody” is used a subset of “chimeric antibodies.”

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR (hereinafter defined) of the recipient are replaced by residues from an HVR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al, Nature 321:522-525 (1986); Riechmann et al, Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol, 227:381 (1991); Marks et al, J. Mol. Biol, 222:581 (1991). Also available for the preparation of human monoclonal antibodies are methods described in Cole et al, Monoclonal Antibodies and Cancer Therapy, Alan R. Liss, p. 77 (1985); Boerner et al, J. Immunol, 147(I):86-95 (1991). See also van Dijk and van de Winkel, Curr. Opin. Pharmacol, 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al, Proc. Natl. Acad. Sci. USA, 103:3557-3562 (2006) regarding human antibodies generated via a human B-cell hybridoma technology.

Avelumab (formerly designated MSB0010718C) is a fully human monoclonal antibody of the immunoglobulin (Ig) G1 isotype. Avelumab selectively binds to PD-L1 and competitively blocks its interaction with PD-1.

Compared with anti-PD-1 antibodies that target T-cells, Avelumab targets tumor cells, and therefore is expected to have fewer side effects, including a lower risk of autoimmune-related safety issues, as blockade of PD-L1 leaves the PD-L2-PD-1 pathway intact to promote peripheral self-tolerance (Latchman Y, Wood C R, Chernova T, et al. PD-L1 is a second ligand for PD-1 and inhibits T cell activation. Nat Immunol 2001; 2(3):261-68).

Avelumab, its sequence and many of its properties have been described in WO2013079174, where it is designated A09-246-2, having the heavy chain and light sequences according to SEQ ID NOs: 32 and 33, as shown in FIG. 1 (SEQ ID NO:7) and FIG. 2 (SEQ ID NO:9), of this patent application. As shown in WO2013079174, one of Avelumab's properties is its ability to exert antibody-dependent cell-mediated cytotoxicity (ADCC), thereby directly acting on PD-L1 bearing tumor cells by inducing their lysis without showing any significant toxicity.

Typically, the inhibitors, e.g. antibodies or antibody fragments according to the invention are incorporated into pharmaceutical compositions suitable for administration to a subject, wherein the pharmaceutical composition comprises the inhibitors, e.g. antibodies or antibody fragments and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof.

In many cases, it is preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the inhibitors, e.g. antibodies or antibody fragments.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans. The preferred mode of administration is parenteral (e. g., intravenous, subcutaneous, intraperitoneal, intramuscular). In a preferred embodiment, the inhibitor, e.g. antibody or antibody fragment is administered by intravenous infusion or injection. In another preferred embodiment, the inhibitor, e.g. antibody or antibody fragment is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i. e., inhibitor, e.g. antibody or antibody fragment) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

A “therapeutically effective amount” of an inhibitor, e.g. antibody or antibody fragment of the invention refers to an amount effective, at dosages and for periods of time necessary, to achieve the desired therapeutic result. Such therapeutically effective amount may vary according to factors such as the disease state, age, sex, and weight of the individual, and the ability of the inhibitor, e.g. antibody or antibody fragment to elicit a desired response in the individual. A therapeutically effective amount is also one in which any toxic or detrimental effects of the inhibitor, e.g. antibody or antibody fragment are outweighed by the therapeutically beneficial effects.

“Chemotherapy” is a therapy involving a “chemotherapeutic agent”, which is a chemical compound useful in the treatment of cancer. Examples of chemotherapeutic agents include alkylating agents such as thiotepa and cyclophosphamide; alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol); beta-lapachone; lapachol; colchicines; betulinic acid; a camptothecin (including the synthetic analogue topotecan (CPT-11 (irinotecan), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gammall and calicheamicin omegall (see, e.g., Nicolaou et ah, Angew. Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, doxorubicin HCl liposome injection and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine, tegafur, capecitabine, an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-Kit inhibitors; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); thiotepa; taxoids, e.g., paclitaxel, albumin-engineered nanoparticle formulation of paclitaxel, and doxetaxel; chloranbucil; 6-thioguanine; mercaptopurine; methotrexate; platinum analogs such as cisplatin and carboplatin; vinblastine; platinum; etoposide (VP-16); ifosfamide; mitoxantrone; vincristine; oxaliplatin; leucovovin; vinorelbine; novantrone; edatrexate; daunomycin; aminopterin; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin combined with 5-FU and leucovovin.

“Platinum-based chemotherapy” as used herein refers to therapy with one or more platinum-based chemotherapeutic agents, optionally in combination with one or more other chemotherapeutic agents.

The phrase “progressed after chemotherapy” refers to progression of the carcinoma while receiving chemotherapy (i.e. refractory) or progression of the carcinoma within 12 months (e.g. within 6 months) after completing the chemotherapy regimen.

“Objective response” refers to a measurable response, including complete response (CR) or partial response (PR).

“Complete response” or “complete remission” refers to the disappearance of all signs of cancer in response to treatment. This does not always mean the cancer has been cured.

“Partial response” refers to a decrease in the size of one or more tumors or lesions, or in the extent of cancer in the body, in response to treatment.

A “PD-L1 positive” cancer is one comprising cells which have PD-L1 present at their cell surface. Preferably, the cancer is “PD-L1 positive” according to the invention, when between at least 0.1% and at least 10% of the cells of the cancer have PD-L1 present at their cell surface. More preferably, the cancer is “PD-L1 positive”, when between at least 0.5% and 5% of the cells of the cancer have PD-L1 present at their cell surface. Most preferably, the cancer is “PD-L1 positive”, when at least 1 of the cells of the cancer have PD-L1 present at their cell surface.

The term “PD-L1 positive” also refers to a cancer that produces sufficient levels of PD-L1 at the surface of cells thereof, such that an anti-PD-L1 inhibitor (e.g. antibody) has a therapeutic effect, mediated by the binding of the said anti-PD-L1 inhibitor (e.g. antibody) to PD-L1.

In a preferred embodiment the PD-L1 expression is determined by immunohistochemistry (IHC).

“Advanced” cancer is one which has spread outside the site or organ of origin, either by local invasion or metastasis. Accordingly, the term “advanced” cancer includes both locally advanced and metastatic disease.

“Recurrent” cancer is one which has regrown, either at the initial site or at a distant site, after a response to initial therapy, such as surgery. A “locally recurrent” cancer is cancer that returns after treatment in the same place as a previously treated cancer.

“Unresectable” cancer is not able to be removed (resected) by surgery.

“Metastatic” cancer refers to cancer which has spread from one part of the body (e.g. the lung) to another part of the body.

“Locally advanced” cancer refers to cancer that has spread to nearby tissues or lymph nodes, but not metastasized.

“Advanced unresectable” cancer is one which has spread outside the site or organ of origin, either by local invasion or metastasis and which is not able to be removed (resected) by surgery.

“Subject” includes a human patient. The patient may be a “cancer patient,” i.e. one who is suffering or at risk for suffering from one or more symptoms of cancer, in particular non-small cell lung cancer.

“Infusion” or “infusing” refers to the introduction of a drug-containing solution into the body through a vein for therapeutic purposes. Generally, this is achieved via an intravenous (IV) bag.

“Systemic treatment” is a treatment wherein the drug substance travels through the bloodstream, reaching and affecting cells all over the body.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

“Antibody-dependent cell-mediated cytotoxicity” or ADCC refers to a form of cytotoxicity in which secreted Ig bound onto Fc receptors (FcRs) present on certain cytotoxic cells (e.g., natural killer (NK) cells, neutrophils and macrophages) enable these cytotoxic effector cells to bind specifically to an antigen-bearing target cell and subsequently kill the target cell with cytotoxins. The antibodies “arm” the cytotoxic cells and are required for killing of the target cell by this mechanism. The primary cells for mediating ADCC, NK cells, express FcγRIII only, whereas monocytes express FcγRI, FcγRII and FcγRIII. Fc expression on hematopoietic cells is summarized in Table 3 on page 464 of Ravetch and Kinet, Annu. Rev. Immunol. 9: 457-92 (1991).

### Specific Description of the Invention

### Ovarian Cancer

In one specific aspect the invention provides a method of treating ovarian cancer in a subject, comprising administering to the subject a therapeutically effective amount of an inhibitor of the interaction between the PD-1 receptor and its ligand PD-L1.

In one embodiment of this aspect the subject in which ovarian cancer is treated is human, the PD-1 receptor is human PD-1 receptor, and PD-L1 is human PD-L1.

In one embodiment the inhibitor binds to PD-L1. Preferably, the inhibitor is an anti-PD-L1 antibody, or an antigen binding fragment thereof. More preferably, the anti-PD-L1 antibody, or an antigen binding fragment thereof, comprises

In its heavy chain the three complementarity determining regions (CDR's) according to SEQ ID NO's 1, 2 and 3, and in its light chain the three complementarity determining regions (CDR's) according to SEQ ID NO's 4, 5 and 6. Most preferably the anti-PD-L1 antibody is Avelumab, having the heavy and light chain sequences shown in FIG. 1a or 1b and 2 (SEQ ID NO's 7 or 8 and 9), or an antigen binding fragment thereof.

In one embodiment the subject having ovarian cancer has not been previously treated for ovarian cancer.

In one embodiment the subject having previously untreated ovarian cancer is receiving the inhibitor in combination with chemotherapy.

In one embodiment said combination therapy is simultaneous. In another embodiment said combination therapy is sequential.

In one embodiment the subject having previously untreated ovarian cancer is receiving the inhibitor following chemotherapy.

In a preferred embodiment said chemotherapy is platinum-based chemotherapy.

In one embodiment the ovarian cancer is identified as a PD-L1 positive cancer.

In one embodiment the inhibitor is an anti-PD-L1 antibody, which is administered at a dose of approximately 10 mg/kg body weight every other week.

In one embodiment the anti-PD-L1 antibody is administered as an intravenous infusion or subcutaneously.

In one embodiment the anti-PD-L1 antibody is administered as a one hour intravenous infusion.

In one embodiment the method results in an objective response, preferably a complete response or a partial response.

### Renal Cell Carcinoma

In one specific aspect the invention provides a method of treating renal cell carcinoma in a subject, comprising administering to the subject a therapeutically effective amount of an inhibitor of the interaction between the PD-1 receptor and its ligand PD-L1.

In one embodiment of this aspect the subject in which renal cell carcinoma is treated is human, the PD-1 receptor is human PD-1 receptor, and PD-L1 is human PD-L1.

In one embodiment the inhibitor binds to PD-L1. Preferably, the inhibitor is an anti-PD-L1 antibody, or an antigen binding fragment thereof. More preferably, the anti-PD-L1 antibody, or an antigen binding fragment thereof, comprises

In its heavy chain the three complementarity determining regions (CDR's) according to SEQ ID NOs: 1, 2 and 3, and in its light chain the three complementarity determining regions (CDR's) according to SEQ ID NOs: 4, 5 and 6. Most preferably the anti-PD-L1 antibody is Avelumab, having the heavy and light chain sequences shown in FIG. 1a or 1b and 2 (SEQ ID NOs: 7 or 8 and 9), or an antigen binding fragment thereof.

In one embodiment the subject having the metastatic renal cell carcinoma, has previously received systemic treatment.

In one embodiment the renal cell carcinoma is treated with the inhibitor as a single agent.

In one embodiment the renal cell carcinoma is identified as a PD-L1 positive cancer.

In one embodiment the inhibitor is an anti-PD-L1 antibody, which is administered at a dose of approximately 10 mg/kg body weight every other week.

In one embodiment the anti-PD-L1 antibody is administered as an intravenous infusion or subcutaneously.

In one embodiment the anti-PD-L1 antibody is administered as a one hour intravenous infusion.

In one embodiment the method results in an objective response, preferably a complete response or a partial response.

### Hodgkin's Lymphoma

Previous studies by others indicated that PD-L1 and PD-L2 transcripts are abundant in Hodgkin's Lymphoma (HL) cell lines. HL cells lines with increased copies of 9p24.1 had significantly higher cell-surface expression of the PD-L1 and PD-L2 proteins. It has been generally believed that in order to treat Hodgkin's Lymphoma, it is necessary to block both the PD-L1/PD-1 interaction and the PD-L2/PD-1 interaction. (M. Shipp et al, Blood, Vol 116, No. 17, 2010) It was surprisingly found out, that Avelumab, being a PD-L1 inhibitor, without known binding affinity to PD-L2 (Kd>1 μM), demonstrated efficacy in patients with classical Hodgkin's Lymphoma.

In one specific aspect the invention provides a method of treating Hodgkin's lymphoma in a subject, comprising administering to the subject a therapeutically effective amount of an inhibitor of the interaction between the PD-1 receptor and its ligand PD-L1. Preferably, the inhibitor is an anti-PD-L1 antibody that binds to human PD-L2 at an affinity of at least 10 times, 100 times, 1000 times, 10⁴ times, 10⁵ times or 10⁶ times lower than it binds to human PD-L1. Even more preferably, the inhibitor is an anti-PD-L1 antibody that binds to human PD-L2 at an affinity of at least 1000 times lower than it binds to human PD-L1.

In one embodiment of this aspect the subject in which Hodgkin's lymphoma is treated is human, the PD-1 receptor is human PD-1 receptor, and PD-L1 is human PD-L1.

In one embodiment the inhibitor binds to PD-L1. Preferably, the inhibitor is an anti-PD-L1 antibody, or an antigen binding fragment thereof. More preferably, the anti-PD-L1 antibody, or an antigen binding fragment thereof, comprises

In its heavy chain the three complementarity determining regions (CDR's) according to SEQ ID NOs: 1, 2 and 3, and in its light chain the three complementarity determining regions (CDR's) according to SEQ ID NOs: 4, 5 and 6. Most preferably the anti-PD-L1 antibody is Avelumab, having the heavy and light chain sequences shown in FIG. 1a or 1b and 2 (SEQ ID NOs: 7 or 8 and 9), or an antigen binding fragment thereof.

In one embodiment the Hodgkin's lymphoma is classical Hodgkin's lymphoma.

In one embodiment the Hodgkin's lymphoma is advanced stage.

In one embodiment the subject has previously received chemotherapy.

In one embodiment the inhibitor is an anti-PD-L1 antibody, which is administered at a dose of approximately 10 mg/kg body weight every other week.

In one embodiment the anti-PD-L1 antibody is administered as an intravenous infusion or subcutaneously.

In one embodiment, the Hodgkin's lymphoma is classical Hodgkin's lymphoma and the subject underwent allogeneic stem cell transplantation prior to the administration of the inhibitor.

In one aspect of this embodiment, the subject underwent allogeneic stem cell transplantation at least six month prior, and preferably at least twelve months prior to the administration of the inhibitor. More preferably the subject underwent allogeneic stem cell transplantation between six months to five years, six months to four years, six months to three years, or six months to two years prior to the administration of the inhibitor.

In another aspect of this embodiment, the subject does not have a medical history suggesting significant risk of serious graft-versus-host-disease upon the administration of the anti-PD-L1 antibody. More specifically, the subject did not receive immunosuppressive treatment for acute or chronic graft-versus-host disease (GVHD) within 3 months prior to administration of the inhibitor; did not have grade 3 or grade 4 GVHD at any time; did not at any time have chronic GVHD persisting for more than 6 months and requiring systemic immunosuppression; and/or did not receive a donor lymphocyte infusion (DLI) within 6 month prior to administration of the inhibitor.

In another aspect of this embodiment, the inhibitor is Avelumab, an anti-PD-L1 antibody, and that the subject is administered Avelumab intravenously at a dosing of 10-20 mg/kg every two weeks, 70-500 mg flat dose every two weeks or 70-500 mg flat dose every three weeks. Preferably the dosing is at least 70 mg every two weeks. More preferably, the dosing is 70 mg every two weeks, 350 mg every two weeks or 500 mg every two weeks. Preferably, the subject is undergoing treatment of Avelumab for a period that the subject receives at least one dose, at least two doses, at least three doses or at least 4 doses of Avelumab.

In one embodiment the anti-PD-L1 antibody is administered as a one hour intravenous infusion.

### Abbreviations

AE Adverse event

### Allo-SCT Allogeneic Stem Cell Transplantation

### AUC Area Under Curve

### Av Avelumab

BOR Best overall response
CR Complete response

### CTCAE Common Terminology Criteria for Adverse Events

### ECOG Eastern Cooperative Oncology Group

EGFR Epidermal growth factor receptor

### EORTC European Organization for Research and Treatment of Cancer

EQ-5D EuroQOL 5-dimensions questionnaire

### GVHD Graft-Versus-Host Disease

### IERC Independent Endpoint Review Committee

### IHC Immunohistochemistry

### IV Intravenous

### ITT Intention To Treat

### LA Locally Advanced

NSCLC Non-small cell lung cancer
ORR Objective response rate
OS Overall survival

### pCR Pathologic Complete Response

### PD Progressive Disease

PFS Progression-free survival
PFS2 Time to second objective disease progression
PR Partial response

### QLQ-LC13 Quality of Life Questionnaire-Lung Cancer

Q2W Every second week
Q3W Every third week

### RECIST 1.1 Revised Guidelines for Response Evaluation Criteria in Solid Tumors

SAE Serious adverse event

### SD Stable Disease

### SOC Standard Of Care

### TEAE Treatment-Emergent Adverse Event

### Example 1

This example is about an open-label, multicenter, three-arm phase III trial testing Avelumab in combination with and/or following platinum-based chemotherapy in patients with previously untreated ovarian cancer.

The primary objective is to demonstrate that Avelumab in combination with and/or following frontline chemotherapy is superior to chemotherapy alone followed by observation in progression-free survival (PFS) by central review. Eligibility criteria include newly diagnosed stage III-IV epithelial ovarian, fallopian tube, or primary peritoneal cancer following debulking surgery or prior to neoadjuvant chemotherapy, irrespective of PD-L1 status. Chemotherapy backbone allows a choice of weekly (80 mg/m2) or Q3W (175 mg/m2) paclitaxel with Q3W (every three weeks) carboplatin. Approximately 951 eligible patients will be randomized to receive chemotherapy followed by observation; chemotherapy followed by Avelumab; or chemotherapy+avelumab followed by Avelumab. Avelumab is administered at 10 mg/kg Q3W with chemotherapy. Maintenance is at 10 mg/kg Q2W for a maximum of 24 months. Neoadjuvant patients in each arm will undergo interval debulking after 3 cycles. Secondary endpoints include overall survival, PFS by gynecological cancer intergroup criteria, maintenance PFS pCR, PFS2, pharmacokinetics, immunogenicity, quality of life, safety, and biomarkers in tumor and blood.

### Example 2

This example is about a phase Ib trial testing Avelumab in patients with metastatic renal cell carcinoma.

Eligible patients had histologically confirmed mRCC with a clear-cell component, measurable disease, available archival/fresh tumor biopsy, and an ECOG performance score of 0-1. Initial pts were also required to have failed 1 prior systemic therapy for mRCC. Patients received Avelumab 10 mg/kg (1 h IV infusion) Q2W until confirmed progression, unacceptable toxicity, or withdrawal. Tumors were assessed every 6 weeks by RECIST 1.1 and adverse events (AEs) were graded by NCI-CTCAE v4.0.

By data cut-off, 19 patients had been treated with Avelumab for a median of 20 weeks (range, 2-32) and followed for 3 weeks. Median age was 69 years (range, 30-80) and 15 patients (78.9%) were male. Median time since metastatic diagnosis was 14.7 months, and patients had received a median of 1 prior line (range, 1-5) for advanced disease, including a kinase inhibitor in 9 patients (47.4%) and chemotherapy in 8 patients (42.1%). During Avelumab treatment, 14 patients (73.7%) had a treatment-related (TR) AE; only fatigue (5 patients [26.3%]) and infusion-related reaction (5 patients [26.3%]) occurred in ≥10% of patients. Only 1 patient (5.3%) had a grade 3 TRAE (fatigue), and no grade 4 TRAEs or treatment-related deaths occurred. Unconfirmed overall response rate was 10.5% (95% CI: 1.3, 33.1) based on 2 partial responses; both were ongoing at last evaluation. 14 additional patients (73.7%) had stable disease, resulting in a disease control rate of 84.2%. Median progression-free survival was not reached; 12-week rate was 64.9% (95% CI: 38.0, 82.5).

### Conclusions

Single-agent Avelumab has antitumor activity and a manageable safety profile in patients with mRCC in the second-line setting. Based on responses observed, this cohort has been expanded to enroll >30 patients with mRCC receiving first-line Avelumab.

### Example 3

This example is about a phase I pharmacokinetic—pharmacodynamic study of Avelumab in previously treated, advanced stage classical Hodgkin's lymphoma.

The study is a Phase 1b dose-finding study to evaluate the pharmacokinetic, pharmacodynamic, and preliminary antitumor activity of Avelumab in adult patients with cHL. Patients enrolled in the study are required to have failed a first-line salvage chemotherapy regimen. The treatment cohorts will explore factors of nominal dose, dosing frequency, and weight based versus fixed dosing. In the lead-in, a total of N=30 patients will be randomized (1:1) across 5 treatment cohorts. Up to 3 treatment cohorts will be expanded in a dose-expansion where up to N=36 additional patients will be randomized (1:1). Selection criteria for dose expansion cohorts include: safety, achieving >90% mean target occupancy (TO) and observing ≥3 confirmed objective responses per Response Criteria for Malignant Lymphoma. Biomarker evaluation will be performed to assess target expression, phenotypes of infiltrating immune cells, and markers associated with immune activation and tolerance along with levels of cytokines, chemokines, and soluble receptors associated with immune regulation. This investigation will define Avelumab pharmacokinetic parameters, confirm TO, and identify pharmacodynamic effects and/or immunophenotypes associated with tumor and clinical response in patients with cHL. It will also establish the functional relevance of PD-L2 in driving the disease phenotype.

As of March 2017, 31 patients were dosed for a period of at least two weeks, but preferably more than 6 weeks, to allow us to evaluate the efficacy of the drug. Six of the thirty-one patients treated had received prior allogeneic stem cell transplantation (post-allo SCT). Patients were treated with Avelumab at one of the following dosing regimens: 70 mg Avelumab Q2W, 350 mg Avelumab Q2W, 500 mg Avelumab Q3W, 500 mg Avelumab Q2W, and 10 mg/kg Avelumab Q2W. Patient response are indicated in the following Tables 1 (all patients) and 2 (post-allo SCT patients).

TABLE 1
All Patient Response

| | 70 mg | 350 mg | 500 mg | 500 mg | 10 mg/kg | |
|---|---|---|---|---|---|---|
| | Q2W | Q2W | Q3W | Q2W | Q2W | Total |
| | (N = 6) | (N = 7) | (N = 6) | (N = 7) | (N = 6) | (N = 31) |
| CR (n) | 1 | 0 | 1 | 0 | 0 | 2 (6.5%) |
| PR (n) | 3 | 1 | 4 | 3 | 4 | 15 (48.4%) |
| ORR | 66.7% | 14.3 | 83.3% | 50% | 66.7% | 17 (54.8%) |

TABLE 2
Response of Post-allo SCT Patients *

| | 70 mg | 350 mg | 500 mg | 500 mg | 10 mg/kg | |
|---|---|---|---|---|---|---|
| | Q2W | Q2W | Q3W | Q2W | Q2W | Total |
| | (N = 1) | (N = 2) | (N = 2) | (N = 2) | (N = 1) | (N = 6) |
| CR (n) | 0 | 0 | 1 | 0 | 0 | 1 (12.5%) |
| PR (n) | 1 | 0 | 4 | 2 | 1 | 5 (62.5%) |
| ORR | 100% | 0 | 100% | 100% | 100% | 6 (75%) |

Notes:
* Patients had received allogeneic stem cell transplantation prior to the administration of Avelumab.

One patient showed a complete response (CR); this patient had been treated with 500 mg Avelumab Q3W and had previously received an allogeneic stem cell transplantation. Patients showing a partial response (PR) included: three patients who had received 70 mg Avelumab Q2W, one patient who had received 350 mg Avelumab Q2W, four patients who had received 500 mg Avelumab Q3W, three patients who had received 500 mg Avelumab Q2W, and four patients who had received 10 mg/kg Avelumab Q2W.

Patients who had received allogeneic stem cell transplantation prior to the administration of Avelumab had 75% overall response rate (ORR) and 12.5% complete response rate (CR) and 62.5% partial response rate (PR) (Table 2). By comparison, response rate in all patients were as follows: 54.8% ORR, 6.5% CR and 54.8% PR.

It is noted that one post-allo SCT patient achieved complete response after only one dose of Avelumab at 500 mg. The patient developed GVHD after the first dose of Avelumab, and the patient did not receive additional doses of Avelumab. The GVHD was subsequently controlled.

### Example 4

This example is about a phase Ib trial testing Avelumab in patients with platinum-refractory or platinum-ineligible metastatic head and neck squamous cell carcinoma (HNSCC).

Patients with platinum-refractory or platinum-ineligible, human papillomavirus-positive or negative, metastatic HNSCC received Avelumab 10 mg/kg (1 h IV) Q2W until confirmed progression, unacceptable toxicity, or withdrawal. Tumors were assessed every 6 weeks (RECIST v1.1 by independent review). Endpoints included objective response rate (ORR), progression-free survival (PFS) and safety (NCI-CTCAE v4.0).

As of Dec. 18, 2015, 153 patients had been treated with Avelumab. Primary tumor sites were oral cavity (28.1%), oropharynx (21.6%), hypopharynx (13.1%), larynx (10.5%), other (25.5%), or missing (1.3%). Median time from metastatic diagnosis was 13.7 months. 48.3% had received ≥2 prior lines for advanced disease (range 0-6). Median duration of treatment was 11.9 weeks (range 2-34). 79 patients (51.6%) had a treatment-related (TR) AE; most common (≥6%) were fatigue (9.8%), pyrexia (9.2%), and infusion-related reaction (8.5%). 8 patients (5.2%) had a grade 3-4 TRAE. 5 patients (3.3%) had an immune-mediated TRAE, including 1 grade 3 (psoriasis). There were no treatment-related deaths. Among 90 patients with ≥3 months follow-up, unconfirmed ORR was 12.2% (95% CI 6.3, 20.8) based on 11 partial responses; 9/11 (81.8%) were ongoing at cutoff. 28 patients (31.1%) had stable disease. Based on a ≥5% PD-L1 staining threshold (76/90 evaluable), ORR in PD-L1+ and PD-L1− tumors was 9.8% (5/51; 95% CI: 3.3, 21.4) and 16.0% (4/25; 4.5, 36.1). Median PFS was 7.7 weeks (95% CI 6.0, 11.7) in all treated patients, and 6.0 vs. 6.4 weeks in evaluable patients with PD-L1+ or PD-L1− tumors.

### CONCLUSIONS

Avelumab showed promising clinical activity and was well tolerated in patients with platinum-refractory or platinum-ineligible HNSCC.

1. A method of treating cancer in a subject, comprising administering to the subject a therapeutically effective amount of an inhibitor of the interaction between the PD-1 receptor and its ligand PD-L1.

2. The method according to claim 1, wherein the cancer is ovarian cancer, renal cell carcinoma, Hodgkin's lymphoma, or head and neck squamous cell carcinoma (HNSCC).

3. The method according to claim 1, wherein the subject is human, the PD-1 receptor is human PD-1 receptor, and PD-L1 is human PD-L1.

4. The method according to claim 1, wherein the inhibitor binds to PD-L1.

5. The method according to claim 1, wherein the cancer is identified as a PD-L1 positive cancer.

6. The method according to claim 4, wherein the inhibitor is an anti-PD-L1 antibody.

7. The method according to claim 6, wherein the anti-PD-L1 antibody comprises in its heavy chain the three complementarity determining regions (CDRs) according to SEQ ID NOs: 1, 2 and 3, and in its light chain the three complementarity determining regions (CDRs) according to SEQ ID NOs: 4, 5 and 6.

8. The method according to claim 6, wherein the anti-PD-L1 antibody is Avelumab, having the heavy chain sequences according to SEQ ID NOs: 7 or 8 and the light chain sequence according to SEQ ID NO:9.

9. The method according to claim 6, wherein the anti-PD-L1 antibody is administered at a dose of 10 mg/kg body weight every other week.

10. The method according to anyone of claim 6, wherein the anti-PD-L1 antibody is administered as an intravenous infusion or subcutaneously.

11. The method according to claim 10, wherein the anti-PD-L1 antibody is administered as a one hour intravenous infusion.

12. The method according to claim 1, wherein the method results in an objective response, preferably a complete response or a partial response.

13. The method according to claim 1, wherein the inhibitor is administered as a single agent, not as part of a combination therapy.

14. The method according to claim 1, wherein the subject has previously received cancer treatment.

15. The method according to claim 14, wherein the cancer treatment is chemotherapy.

16. The method according to claim 15, wherein the chemotherapy comprises a platinum containing chemotherapeutic agent.

17. The method according to claim 16, wherein the chemotherapy is platinum-containing doublet chemotherapy.

18. The method according to claim 2, wherein the cancer is ovarian cancer.

19. The method according to claim 18, wherein the ovarian cancer has not previously been treated.

20. The method according to claim 18, wherein the ovarian cancer is treated with a combination of the said inhibitor and chemotherapy.

21. The method according to claim 18, wherein the ovarian cancer is treated with the said inhibitor following chemotherapy.

22. The method according to claim 20, wherein chemotherapy is platinum-based chemotherapy.

23. The method according to claim 2, wherein the cancer is renal cell carcinoma.

24. The method according to claim 23, wherein the renal cell carcinoma is metastatic renal cell carcinoma.

25. The method according to claim 24, wherein the metastatic renal cell carcinoma has previously received systemic treatment.

26. The method according to claim 2, wherein the cancer is Hodgkin's lymphoma.

27. The method according to claim 26, wherein the inhibitor is an anti-PD-L1 antibody that binds to human PD-L2 at an affinity of at least 10 times, 100 times, 1000 times, 10⁴ times, 10⁵ times or 10⁶ times lower than it binds to human PD-L1.

28. The method according to claim 26, wherein the Hodgkin's lymphoma is classical Hodgkin's lymphoma.

29. The method according to claim 26, wherein the Hodgkin's lymphoma is advanced stage.

30. The method according to claim 26, wherein the Hodgkin's lymphoma has previously received chemotherapy.

31. The method according to claim 28, wherein the subject underwent allogeneic stem cell transplantation (allo SCT) prior to the administration of the inhibitor.

32. The method according to claim 31, wherein the subject underwent allo SCT at least six months prior to the administration of the inhibitor.

33. The method according to claim 32, wherein the subject underwent allo SCT between six months to five years prior to the administration of the inhibitor.

34. The method according to claim 31, wherein the subject did not receive immunosuppressive treatment for acute or chronic graft-versus-host disease (GVHD) within 3 months prior to administration of the inhibitor; did not have grade 3 or grade 4 GVHD at any time; did not at any time have chronic GVHD persisting for more than 6 months and requiring systemic immunosuppression; and/or did not receive a donor lymphocyte infusion (DLI) within 6 months prior to administration of the inhibitor.

35. The method according to claim 2, wherein the cancer is HNSCC.

36. The method according to claim 35, wherein the HNSCC is metastatic.

37. The method according to claim 35, wherein the HNSCC has previously received chemotherapy comprising a platinum containing chemotherapeutic agent.

38. The method according to claim 37, wherein the HNSCC is platinum-refractory.

39. The method according to claim 35, wherein the HNSCC is platinum-ineligible.

40. The method according to claim 35, wherein the HNSCC is metastatic, and platinum-refractory or platinum-ineligible.


SEQUENCE LISTING (9 sequences)

SEQ ID NO: 1 - protein, 5 aa, Artificial Sequence
  from human Fab library
SYIMM

SEQ ID NO: 2 - protein, 16 aa, Artificial Sequence
  from human Fab library
SIYPSGGITFYADTVK

SEQ ID NO: 3 - protein, 11 aa, Artificial Sequence
  from human Fab library
IKLGTVTTVDY

SEQ ID NO: 4 - protein, 14 aa, Artificial Sequence
  from human Fab library
TGTSSDVGGYNYVS

SEQ ID NO: 5 - protein, 7 aa, Artificial Sequence
  from human Fab library
DVSNRPS

SEQ ID NO: 6 - protein, 10 aa, Artificial Sequence
  from human Fab library
SSYTSSSTRV

SEQ ID NO: 7 - protein, 450 aa, Artificial Sequence
  from human Fab library
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 449 aa, Artificial Sequence
  from human Fab library
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 216 aa, Artificial Sequence
  from human Fab library
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS